(19) United States
(12) Patent Application Publication
(10) Pub. No.: US 2008/0097585 A1
(43) Pub. Date: Apr. 24, 2008

(54) VALVE REPAIR SYSTEMS AND METHODS

(76) Inventors: Richard Sisken, West Lafayette, IN (US); Michael C. Hiles, Lafayette, IN (US); Neal E. Fearnot, West Lafayette, IN (US); Alan R. Leewood, Lafayette, IN (US)

Correspondence Address: DUNLAP, CODDING & ROGERS, P.C., P.O. BOX 16370, OKLAHOMA CITY, OK, 73113, US

(21) Appl. No.: 11/772,438

(22) Filed: Jul. 2, 2007

Related U.S. Application Data
(60) Provisional application No. 60/806,296, filed on Jun. 30, 2006.

(51) Int. Cl. A61F 2/06

(52) U.S. Cl. 623/1.24

(57) ABSTRACT

Medical device valve repair systems that include a stabilization member are provided. The stabilization member includes an engaging channel to contact the engaging surface of a valve holding valve leaflets in a position such that a tissue repair device may form a coaptation between the valve leaflets.

### CROSS-REFERENCE TO RELATED APPLICATION

This application claims priority to U.S. Provisional Application No. 60/806,296, filed on Jun. 30, 2006, the entire contents of which are hereby incorporated by reference into this disclosure.

### FIELD

Systems and methods for repairing valves in body vessels and other tissues, such as venous and cardiac valves, are described.

### BACKGROUND

Fluid flow within some body vessels is regulated by various membranous valves. Many valves within the body are composed of thin flexible leaflets intended to allow unidirectional fluid flow. That is, most of these valves permit fluid flow in a first direction (antegrade flow) and substantially prevent fluid flow in a second, opposite direction (retrograde flow). When a leaflet becomes damaged or otherwise fails, retrograde fluid flow through the valve may begin to occur. Over time, valve failure can lead to various disease conditions, including venous insufficiency and slow- and/or non-healing ulcers.

For example, within the venous system, bicuspid valves are oriented in the veins to aid in the return of oxygen-poor blood to the heart. The veins of the leg, which carry blood from the parts of the body that are farthest from the heart, are illustrative of venous valve function. When leg muscles are relaxed, the pressure gradient within the venous system allows blood to flow toward the heart. When leg muscles contract, blood is squeezed toward and away from the heart. Blood flowing in one direction forces one valve open, while blood flowing in the opposite direction forces another valve closed to prevent retrograde blood flow. With every contraction and relaxation, deoxygenated blood continues its journey to the heart.

Absence or reduction in muscle contraction can negatively affect this “muscle pump” action, which can, ultimately affect valve function, for example. Extended periods of sitting or standing can cause blood to pool within the veins due to a lack of contraction of the surrounding muscle tissue. The vessel wall is able to support such pooling only for short periods of time. Consequently, the pooling may lead to stretching of the vessel wall and/or damage to the structure of the wall, each of which can negatively impact the ability of a nearby valve to close and prevent retrograde flow through the vessel. In some people and circumstances, chronic venous insufficiency (CVI) may develop.

CVI can cause swelling of the ankles and legs due to the pooled blood and an abnormally high blood pressure within the vessel. The legs of a person afflicted with CVI may feel achy and tired and may even form a leathery texture, flaking, or itching. Progression of CVI may lead to varicose veins and, in some cases, ulcers that prove difficult to heal. It is estimated that over 20 million adults have been diagnosed worldwide with varicose veins, CVI, or both.

One of the more challenging aspects of treating insufficient valves, as seen in CVI, is providing systems that do not require invasive treatments, such as vein stripping or deep vein surgery. Minimally invasive techniques, which have enjoyed wide acceptance by health care providers and patients alike in many specialties, provide less invasive alternatives for the patient and can reduce treatments costs and recovery time. Unfortunately, the existing art provides limited minimally invasive solutions for the repair of valves.

### SUMMARY OF EXEMPLARY EMBODIMENTS

Systems and methods for repairing one or more valves at one or more valve treatment sites within a body vessel are described. A valve repair system according to one exemplary embodiment includes a sheath and a stabilization member that defines an engaging channel and a working channel. An implement is disposed in the working channel. In one exemplary embodiment, the implement includes a functional end with a pair of grasping arms that include structural adaptations to facilitate connecting of two valve leaflets together. The engaging channel in this embodiment terminates at two openings on a distal portion of the engaging member. In use, valve leaflets are stabilized by drawing the leaflets toward the openings by initiating fluid flow through the openings. Once the leaflets are in contact with a distal portion of the engaging member, the implement is activated to secure the leaflets to each other.

A valve repair system according to a second exemplary embodiment includes a sheath and a stabilization member that defines an engaging channel and two working channels. An implement is disposed in each working channel. Once the leaflets are in contact with a distal portion of the engaging member, the implements are used to force the leaflets into contact with each other and a connection between the leaflets is formed using suitable means for connecting leaflets together.

One exemplary method of repairing a valve includes the steps of providing a valve repair system that includes a sheath, a stabilization member that defines an engaging channel and a working channel, and a valve repair device that can be disposed in the working channel. Another step comprises inserting the valve repair system into a body vessel. Another step comprises advancing the valve repair system through the body vessel to a valve treatment site. Another step comprises initiating fluid flow through the engaging channel to draw at least two leaflets of the valve toward a distal portion of the stabilization member. Another step comprises extending the valve repair device beyond the distal end of the stabilization member. Another step comprises activating the valve repair device to secure the valve leaflets to each other. Another step comprises retracting the valve repair system from the body vessel.

Additional understanding of the invention can be obtained with review of the detailed description of exemplary embodiments, below, and the appended drawings illustrating various exemplary embodiments.

### BRIEF DESCRIPTION OF THE DRAWINGS

FIG. 1 is a schematic sectional view of a body vessel and surrounding tissue. The vessel includes two competent valves; one is illustrated in an open configuration and one is illustrated in a closed configuration.

FIG. 2 is a schematic sectional view of a body vessel and surrounding tissue. The vessel includes one competent and one incompetent valve.

FIG. 3 is a perspective view of a valve repair system according to a first exemplary embodiment.

FIG. 4 is a magnified view of the distal end of the valve repair system illustrated in FIG. 3.

FIG. 5 is a partial sectional view of a body vessel in which a valve repair system has been positioned at a valve treatment site.

FIG. 6 is a magnified view of the distal end of a valve repair system according to an alternative embodiment.

### DETAILED DESCRIPTION OF EXEMPLARY EMBODIMENTS

The following detailed description and the appended drawings describe and illustrate exemplary embodiments of the invention solely for the purpose of enabling one of ordinary skill in the relevant art to make and use the invention. As such, the description and illustration of these embodiments are purely exemplary in nature and are in no way intended to limit the scope of the invention, or its protection, in any manner.

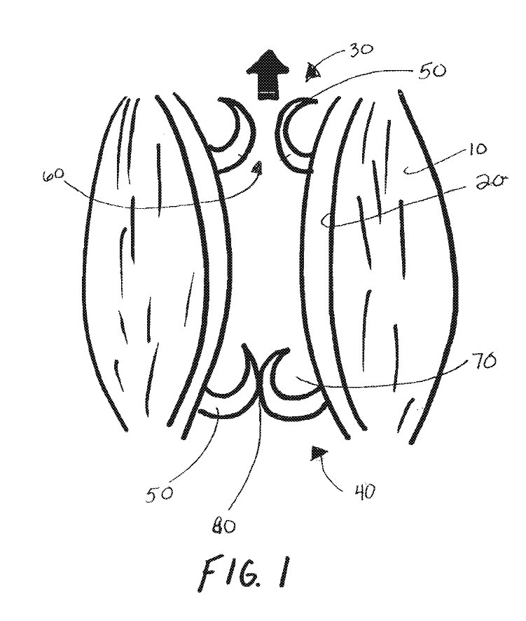

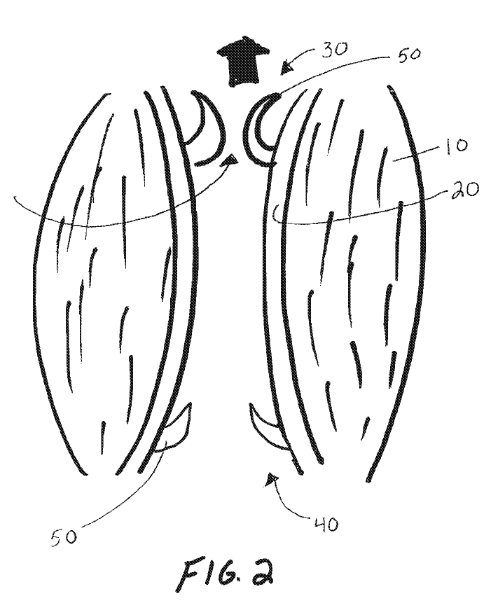

FIGS. 1 and 2 illustrate muscle tissue 10 surrounding a body vessel 20. First 30 and second 40 valves are disposed within the body vessel 20. Each valve 30, 40 includes two valve leaflets 50. When the valve leaflets 50 are in an open position, as illustrated by the first valve 30, a fluid passageway 60 is formed, allowing fluid to flow between the leaflets 50 in substantially one direction (the antegrade direction, represented by the arrow). When the valve leaflets 50 are in a closed position, as illustrated by the second valve 40, the valve leaflets 50 substantially prevent the flow or passage of fluid through a body vessel 20 by forming a reservoir 70 that collects the fluid. As the reservoir 70, frequently referred to as a valve pocket, fills with fluid, the valve leaflets 50 are forced together to form a seal 90 that substantially prevents fluid flow through the body vessel 20 in the retrograde direction (the direction opposite to that represented by the arrow).

FIG. 2 illustrates the effects of a valvular incompetency. The muscles 10 surrounding the body vessel 20, for any of a number of reasons, have allowed the vessel to adopt a widened configuration at the location of the second valve 40. The valve leaflets 50 are unable to form a seal and, as a result, fluid is able to flow through the body vessel 20 in a retrograde direction. The presence of an incompetent valve 50 can lead to various clinical manifestations, including painful, non-healing ulcers in the legs that are commonly associated with venous insufficiency.

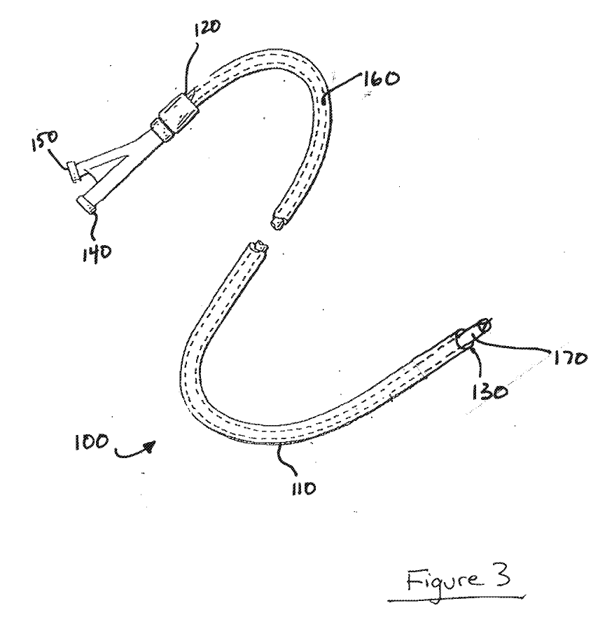

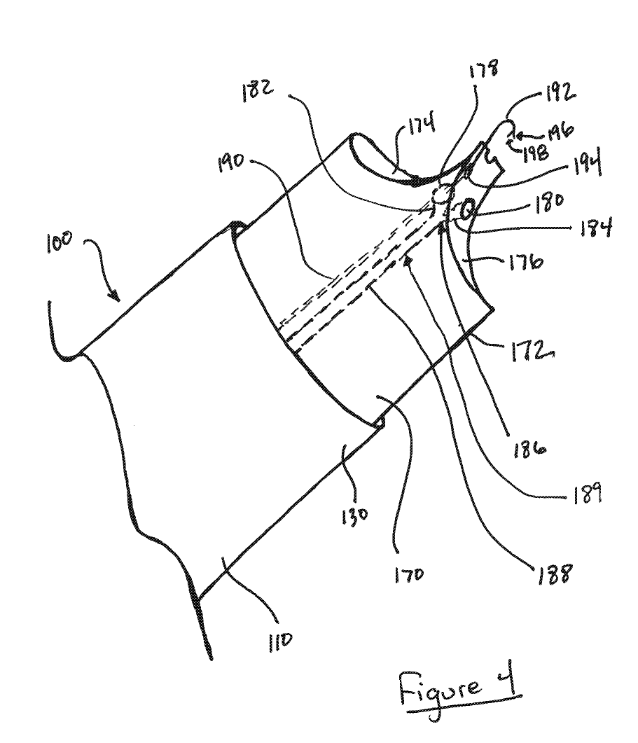

FIGS. 3 and 4 illustrate a valve repair system 100 according to a first exemplary embodiment. The valve repair system 100 includes a sheath 110 having a proximal end 120 and a distal end 130. The sheath 110 is an elongate tubular member that defines a lumen 160 that extends from the proximal end 120 to the distal end 130. The proximal end 120 may include one or more connectors 140, ports 150, or other accessory components for use with the sheath 110. The sheath 110 can be formed from any suitable material. Indeed, standard sheaths typically used in intraluminal medical device delivery systems can be used in a valve repair system according to the invention.

A stabilization member 170 is disposed within the lumen 160 of the sheath 110. The stabilization member 170 is an elongate member having proximal (not shown) and distal 172 ends. The distal end 172 includes first 174 and second 176 receiving surfaces. The first receiving surface 174 defines a first opening 178 and the second receiving surface 176 defines a second opening 180. The first opening 178 provides access to a first sub-channel 182 and the second opening 180 provides access to a second sub-channel 184, each of which is defined by the stabilization member 170. The first 182 and second 184 sub-channels merge at a junction 186 into a main channel 188, which is also defined by the stabilization member 170. The main channel 188 extends from the junction 186 to the proximal end of the stabilization member 170. As such, the main channel 188, in combination with the first 182 and second 184 sub-channels, provides fluid communication between the proximal and distal 172 ends of the stabilization member 170.

The main channel 188, first 182 and second 184 subchannels, and the first 178 and second 180 openings together define an engaging channel 189. Engaging channels in valve repair systems according to other exemplary embodiments can have any suitable structure, configuration and form, and need only provide fluid communication between a distal portion of the stabilization member, such as the receiving surfaces 174, 176, and a proximal portion of the stabilization member so that leaflets of a valve being repaired with the system can be drawn toward the distal portion of the engaging member during stabilization.

As best illustrated in FIG. 4, each receiving surface 174, 176 provides a curvilinear surface. Other configurations could be used, including flat, convex, and concave surfaces. The receiving surfaces 174, 176 need only provide the desired surface to support valve leaflets during a repair procedure, as described in more detail below. The configuration chosen for one or more receiving surfaces in any particular valve repair system according to the invention may depend on several considerations, including the nature of the valve with which the valve repair system is intended to be used.

The stabilization member 170 also defines a working channel 190. The working channel 190 is a lumen within the stabilization member that allows for the insertion and movement of an implement 192 within the stabilization member. In the illustrated embodiment, an opening 194 on the second receiving surface 176 provides access to the working channel 190 from the distal end 172 of the stabilization member 170. Alternatively, the opening 194 could be disposed on the first receiving surface 174. Furthermore, it is expressly understood that additional working channels could be associated with the stabilization member 170. In the illustrated embodiment, the opening 194 is disposed toward one side of the distal end 172 of the stabilization member 170. This arrangement facilitates positioning of the implement 192 adjacent valve leaflets supported on the receiving surfaces 174, 176 during use of the valve repair system 100.

The distal end 172 of the stabilization member 170 defines an edge 197 where the receiving surfaces 174, 176 meet. The edge 197 advantageously defines a notch 199 that provides a void in the support surfaces provided by the receiving surfaces 174, 176. As such, the notch 199 facilitates the securement of valve leaflets to each other using the implement 192, particularly when a clip of other means for securing leaflets to each other is employed.

The implement 192 is an elongate member that can be inserted into and advanced within the working channel 190. The implement 192 defines a functional end 196, such as the grasping arms 198 illustrated in FIG. 4. The functional end 196 includes adaptations to facilitate connecting of two valve leaflets to each other, such as through compression, clamping, securement of a connector (such as an adhesive, a staple, a suture, and the like), or other suitable means for connecting valve leaflets to each other. The grasping arms 198 are just one example of a suitable means for connecting valve leaflets to each other. It is noted that implements with other functionalities can be used with the working channel 190. For example, an endoscopic camera may be advanced through the working channel 180 to enable a practitioner to view the particularities of the body vessel 20 or aid in the repair process. Other exemplary implements include a light, a pharmaceutical delivery device, and the like.

As best illustrated in FIG. 4, the distal end of the implement 192 advantageously adopts a curved or otherwise angled configuration upon exit from the opening 194 of the working channel 190 at the distal end 172 of the engaging member 170. This can be readily accomplished using conventional shape memory materials, including nickel-titanium alloys, and forming techniques. This configuration facilitates interaction between the functional end 196 of the implement 192 and valve leaflets supported on the receiving surfaces 174, 176 of the engaging member 170.

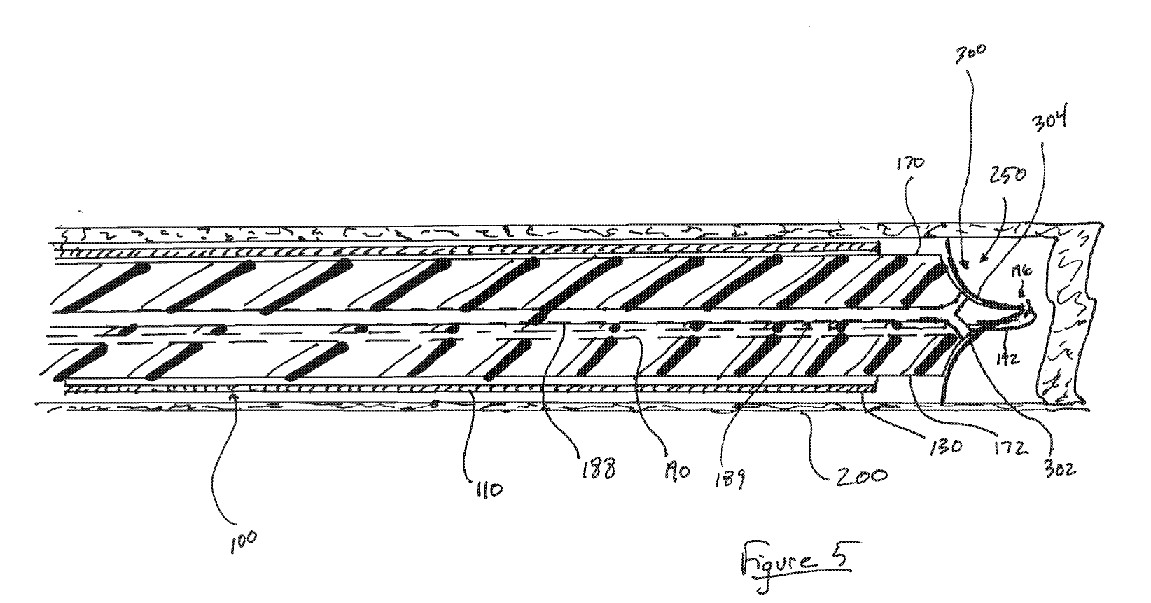

FIG. 5 illustrates the valve repair system 100 in use at a valve treatment site 250 within a body vessel 200. Although the illustrated valve treatment site 250 includes a venous valve 300, it is noted that the valve repair system can be used at any suitable valve treatment site at any valve location, and with any suitable valve, including cardiac valves.

The distal end 130 of the sheath 110 has been advanced proximate to a valve treatment site 250 within the body vessel 200. The sheath 110 may be inserted into a body vessel 200 over a previously placed wireguide using conventional techniques. Alternatively, the sheath 110 can be advanced directly into the body vessel 200 without the use of a wireguide. Appropriate positioning of the sheath 110 at the treatment site 250 may be monitored, determined, and/or confirmed through the use of appropriate imaging equipment and procedures, such as x-ray, ultrasound, and other similar imaging systems.

Standard medical device delivery techniques can be used for advancing the valve repair system 110 to the valve treatment site 140. For example, an introduced sheath (not shown) can be placed in a body vessel 200 such that the distal end of the sheath is at or near the treatment site within the body vessel 200. Once the sheath is positioned in this manner, the valve repair system 110 can be loaded into the proximal end and advanced to the treatment site. In another example, the valve repair system 100 can be used without an introducer sheath and simply be advanced directly over a wireguide using the working channel 190 or a separate wireguide lumen.

The distal end 130 of the sheath 110 is advanced to the valve treatment site 250. Once in this position, the sheath 110 is retracted to expose the distal end 190 of the stabilization member 170. Alternatively, the stabilization member 190 can be advanced while the sheath 110 is held in position, thereby exposing the distal end 190 of the stabilization member at the treatment site. Using either of these exposure techniques, the distal end 190 of the stabilization member, including the engaging surfaces 174, 176, is positioned adjacent a natural valve 300 and the associated leaflets 302, 304.

Fluid flow through the engaging channel 188 is then initiated, such as by activating a pump operably connected to the engaging channel 188, or other suitable means for initiating flow through the channel 188. The means for initiating flow through the channel 188 advantageously creates an area of lower pressure in the engaging channel 188 in relation to the surrounding area, including the receiving surfaces 174, 176. The area of low pressure, and the resulting fluid flow, draws the valve leaflets 302, 304 onto the respective receiving surface 174, 176.

The implement 192 is advanced through the working channel 190 until the functional end 194 is positioned adjacent the coaptation of the valve leaflets 302, 304. Once the implement 192 is positioned in this manner, the functional end 194 is activated to form a connection between the valve leaflets 302, 304. The connection can be formed by an appropriate means for forming a connection, such as staple, fastener, adhesive, clip, magnets, or any other suitable means. A combination of means for forming a connection can also be used. For example, an adhesive can be placed between the valve leaflets 302, 304 before fastening the valve leaflets 302, 304 with a connector, such as a staple or suture. The valve repair system 100 can be removed from the body vessel 200 once the connection is formed.

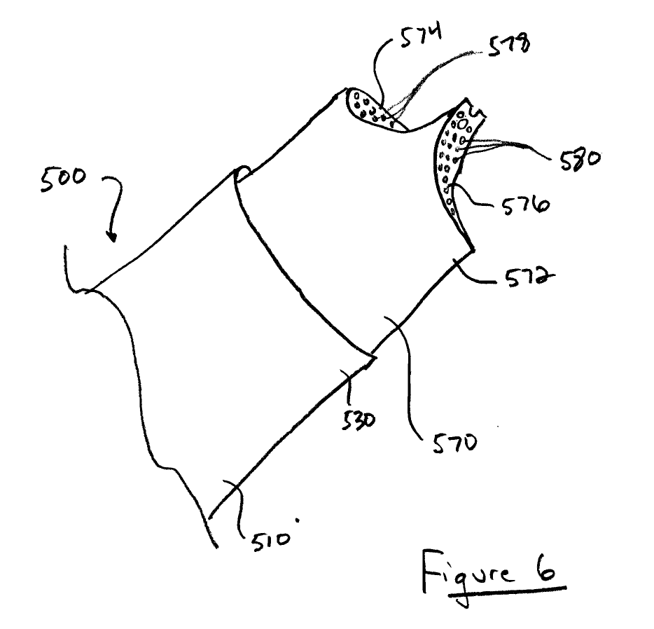

FIG. 6 illustrates a valve repair system 500 according to an alternative embodiment. Similar to the embodiment illustrated in FIGS. 3 and 4, the valve repair system 500 according to this embodiment includes a sheath 510 having a proximal (not illustrated) and distal 530 end and a stabilization member 570 disposed within the lumen defined by the sheath 510. The stabilization member 570 is an elongate member having proximal (not illustrated) and distal 572 ends. The distal end 572 includes first 574 and second 576 receiving surfaces.

In this embodiment, the first receiving surface 574 defines a first plurality of openings 578 that provides access to an engaging channel (not illustrated) defined by the stabilization member, such as the channel described above in regards to the embodiment illustrated in FIGS. 3 and 4. Similarly, the second receiving surface 576 defines a second plurality of openings 580 that provides access to the engaging channel. The use of a plurality of openings disposed substantially over a substantial portion of the receiving surface is considered advantageous at least because this structure is expected to facilitate engagement of valve leaflets during use of the valve repair system 500 by dispersing the attractive force supplied by the engaging channel—such as through fluid flow as described above—over a larger portion of the receiving surfaces 574, 576.

While FIG. 6 illustrates both receiving surfaces 574, 576 as defining a plurality of openings, it is noted that any suitable number can be used and that the surfaces 574, 576 can have different configurations. For example, one receiving surface 574 may define two or fewer openings, while the other receiving surface 576 defines three or more. Any suitable combination of numbers of openings can be used, and the specific structure used for a valve repair system according to a particular embodiment of the invention will depend on various considerations, including the nature and size of the valve(s) with which the system is intended to be used.

Methods of repairing valves are also provided. The methods are suitable for use by health care providers in the various clinical situations, including in the treatment of incompetent venous valves and cardiac valves.

An exemplary method comprises an initial step of providing a valve repair system according to the invention. Another step comprises advancing the valve repair system through a body vessel to a point of treatment. For this step, conventional percutaneous techniques can be used. Another step comprises positioning the receiving surfaces of the engaging member adjacent the valve leaflets of the valve being treated. Another step comprises holding the valve leaflets adjacent the receiving surfaces, such as by initiating fluid flow through a channel that opens to the receiving surfaces. Another step comprises advancing a distal end of an implement out of the working channel and positioning a functional mechanism adjacent the leaflets. Another step comprises forming a connection between the leaflets using the functional mechanism of the implement. Another step comprises withdrawing the valve repair system from the body vessel.

The foregoing detailed description provides exemplary embodiments of the invention and includes the best mode for practicing the invention. The description and illustration of embodiments is intended only to provide examples of the invention and not to limit the scope of the invention, or its protection, in any manner.

We claim:

1. A valve repair system, the system comprising:
an elongate tubular sheath defining a lumen;
a stabilization member disposed in the lumen, the stabilization member having proximal and distal ends and defining an engaging channel and a working channel, the distal end forming first and second valve leaflet receiving surfaces and defining at least one opening to the engaging channel;
an implement disposed in the working channel, the implement including a functional end that includes adaptations to facilitate connecting valve leaflets to each other.

2. The valve repair system according to claim 1, wherein the valve leaflet receiving surfaces comprise curvilinear surfaces.

3. The valve repair system according to claim 1, wherein the engaging channel includes a bifurcation.

4. The valve repair system according to claim 3, wherein the engaging channel includes first and second subchannels, the first subchannel in fluid communication with the first valve leaflet receiving surface and the second subchannel in fluid communication with the second valve leaflet receiving surface.

5. The valve repair system according to claim 1, wherein the functional end includes a pair of grasping arms.

6. The valve repair system according to claim 1, wherein a distal portion of the implement adopts a curved or otherwise angled configuration upon exit from a distal opening of the working channel.

7. The valve repair system according to claim 6, wherein at least the distal portion of the implement is formed of a shape memory material.

8. The valve repair system according to claim 7, wherein the shape memory material comprises a nickel titanium alloy.

9. The valve repair system according to claim 1, wherein at least one of the valve leaflet receiving surfaces defines at least two openings to the engaging channel.

10. The valve repair system according to claim 1, wherein at least one of the valve leaflet receiving surfaces defines a plurality of openings to the engaging channel.

11. The valve repair system according to claim 1, wherein each of the valve leaflet receiving surfaces defines at least two openings to the engaging channel.

12. The valve repair system according to claim 1, wherein each of the valve leaflet receiving surfaces defines a plurality of openings to the engaging channel.

13. The valve repair system according to claim 1, wherein first and second valve leaflet receiving surfaces cooperatively define an edge; and
wherein the edge defines a notch.

14. A valve repair system, comprising:
an elongate tubular sheath defining a lumen;
a stabilization member disposed in the lumen, the stabilization member having proximal and distal ends and defining an engaging channel and first and second working channels, the distal end forming first and second valve leaflet receiving surfaces and defining at least one opening to the engaging channel;
a first implement disposed in the first working channel and a second implement disposed in the second working channel, the first and second implements adapted to cooperatively connect valve leaflets to each other.

15. The valve repair system according to claim 14, wherein at least one of the first and second implements adopts a curved or otherwise angled configuration upon exit from a distal opening of the first or second working channel.

16. The valve repair system according to claim 14, wherein the first implement adopts a curved or otherwise angled configuration upon exit from a distal opening of the first working channel and the second implement adopts a curved or otherwise angled configuration upon exit from a distal opening of the second working channel.

17. A valve repair system, comprising:
an elongate tubular sheath defining a lumen;
a stabilization member disposed in the lumen and including a distal end that defines first and second valve leaflet receiving surfaces, the first valve leaflet receiving surface defining a first opening and the second valve leaflet receiving surface defining a second opening, the stabilization member defining a working channel and an engaging channel comprising a main channel and first and second subchannels, the first subchannel in fluid communication with the first opening and the second subchannel in fluid communication with the second opening;
an implement disposed in the working channel, the implement including a functional end that includes adaptations to facilitate connecting valve leaflets to each other.

18. A method for repairing a valve, the method comprising the steps of:
providing a valve repair system that includes a sheath, a stabilization member that defines an engaging channel and a working channel, and a valve repair device that can be disposed in the working channel;
inserting the valve repair system into a body vessel;
advancing the valve repair system through the body vessel to a valve treatment site;
initiating fluid flow through the engaging channel to draw the valve leaflets toward a distal portion of the stabilization member;
extending the valve repair device beyond the distal end of the stabilization member;
activating the valve repair device to secure the valve leaflets to each other; and
retracting the valve repair system from the body vessel.

19. The method of repairing a valve in claim 18 further comprising the step of forming a coaptation on the valve.

20. The method for repairing a valve of claim 18 wherein the formation of the coaptation is at a midpoint along the width of the valve leaflet.
